(19) United States
(12) Patent Application Publication
(10) Pub. No.: US 2020/0350061 A1
(43) Pub. Date: Nov. 5, 2020

(54) MEDICAL INFORMATION PROCESSING SYSTEM

(71) Applicants: NEC CORPORATION, Tokyo (JP); Kitahara Medical Strategies International Co., Ltd., Tokyo (JP)

(72) Inventors: Masahiro HAYASHITANI, Tokyo (JP); Masahiro KUBO, Tokyo (JP); Shigemi KITAHARA, Tokyo (JP)

(73) Assignee: NEC CORPORATION, Tokyo (JP)

(21) Appl. No.: 16/643,342

(22) PCT Filed: Aug. 22, 2018
(86) PCT No.: PCT/JP2018/030971

§ 371 Date: Feb. 28, 2020

(30) Foreign Application Priority Data
Aug. 30, 2017 (JP) 2017-165607

(51) Int. Cl. G16H 40/20; G16H 50/70; G06N 20/00; G16H 10/60

(52) U.S. Cl. CPC G16H 40/20; G16H 10/60; G06N 20/00; G16H 50/70

(57) ABSTRACT

A medical information processing system comprises: an input unit that receives input of electronic chart information for a patient being treated; a machine learning unit that refers to an electronic chart information group for each patient, the information group being obtained on the basis of an inpatient of an acute care facility, and performs machine learning about the discharge destination, from the acute care facility, of each patient; and a discharge-destination prediction unit that predicts the discharge destination of the patient being treated from the received electronic chart information of the patient being treated, on the basis of the learning results obtained from the machine learning unit.

### TECHNICAL FIELD

This invention relates to a medical information system, a medical information processing method, and a program therefor, which are to be implemented in an acute care facility.

### BACKGROUND ART

Recently, many information processing systems have been used in medical institutions, and information processing systems specifically designed for medical institutions have also been actively developed.

At medical institutions, a wide range of operational tasks including surgery, examinations, and rehabilitation are performed on many patients. The information processing systems specifically designed for medical institutions support the conventional operational tasks performed by medical personnel and improve work efficiency.

Examples of the information processing systems specifically designed for medical institutions include systems configured to convert a conventional paper-based medical chart into an electronic medical chart, and systems configured to receive medical chart information as electronic data from the beginning.

Electronic medical chart information of each patient is stored in a server (storage) of the information processing system, and is called by an authorized staff member of the medical institution to be used as necessary in the same manner as a conventional paper-based medical chart. An information processing system that handles only the collection and presentation of electronic medical charts is generally called an electronic medical chart system.

The medical information systems used in medical institutions are not limited to electronic medical chart systems. Examples of a wide variety of such medical information systems include one system described in Patent Document 1.

In Patent Document 1, there is described a stroke diagnosis collaboration system. This stroke diagnosis collaboration system stores a medical plan for each patient in a shared database in a server installed on the Internet, and shares the stored medical plan for each patient with medical personnel from various facilities via a network. According to this configuration, each patient can receive optimal medical care at various facilities widely distributed across a region based on a unified medical plan. In Patent Document 1, as the various facilities widely distributed across a region, there are mentioned an acute care medical facility, a convalescent rehabilitation medical facility, a general medical facility, a nursing facility, and a family doctor. Therefore, for example, for a patient who has been transported in an emergency, after the patient is treated at the acute care medical facility and discharged from hospital, the family doctor of the patient can know the medical plan established at the acute care medical facility. As a result, a good medical environment for patients can be provided.

As described in Patent Document 1, acute care medical facilities are chronically lacking in hospital beds. According to Patent Document 1, it is described that 80% of hospitalizations to acute care medical facilities are emergency hospitalizations. Patients admitted for emergency hospitalization are generally admitted to an acute care medical facility only during the acute treatment period, and are often transitioned back home or to various facilities during the subsequent recovery or rehabilitation treatment period. Meanwhile, there are rare cases in which a patient hospitalized in an emergency continues to stay in the acute care medical facility with no transition destination suitable for the patient. In addition, there are also cases in which a patient who has been urgently hospitalized may be transferred to various facilities with no expectation of recovery.

In view of the current medical facility situations, with the hospitalization of a patient in an acute care medical facility by emergency transport, a common flow of the patient to be moved from the acute care medical facility to a transition destination is as follows. Specifically, (1) a doctor or the like proposes a medical plan for the patient, (2) treatment is performed during the acute care treatment period in accordance with the medical plan, (3) a transition destination is confirmed and determined based on informed consent, and transition schedule arrangements are made with the transition destination facility, and (4) the patient is moved (discharged from hospital) to the transition destination.

From the perspective of the acute care medical facility, there are disadvantages in keeping patients in hospital longer than required. Meanwhile, acute care medical facilities may not allow patients to be discharged earlier without performing the required measures.

A research report by relevant medical personnel is described in Non Patent Document 1. In Non Patent Document 1, the research report describes that there is a strong association between the Berg balance scale (BBS) at two weeks from an onset of a disease and the functional independence measure (FIM) or BBS at the time of discharge from an acute care hospital. The authors of Non Patent Document 1 suggest that when the BBS at two weeks from the onset of a disease is 40 points or more, there is a high likelihood that the patient can be discharged from the acute care hospital directly to his or her home (for home medical care or home rehabilitation).

### PRIOR ART DOCUMENT(S)

### Patent Document(s)

Patent Document 1: JP 2010-9086 A

### Non-Patent Document(s)

Non-Patent Document 1: Masafumi Kubota, Osamu Yamamura, Tadayoshi Nonoyama, Shinichi Sasaki, Seiichiro Shimada, Hisatoshi Baba, Masaru Kuriyama “The Relation between Berg Balance Scale and Discharge home destination of Acute Phase Stroke Patients”, published 2010, Neurological Therapeutics 27, Pages 573-578

### SUMMARY OF THE INVENTION

### Problem to be Solved by the Invention

In the above-mentioned technology described in Patent Document 1, each patient can receive optimal medical care at various facilities widely distributed across a region based on a unified medical plan. However, the matters described in Patent Document 1 are limited to sharing the medical plan.

Meanwhile, the report described in Non Patent Document 1 suggests that it is possible to predict, based on the acquired BBS score at two weeks from the onset of a disease, that there is shown a high likelihood that the patient can be discharged from an acute care hospital directly to his or her home. However, in this suggested method, it is not possible to predict the transition destination from an acute care medical facility in a shorter period than two weeks after the onset of a disease.

It is an object of this invention to solve at least one of the above-mentioned problems, and to provide a medical information processing system configured to predict, with the hospitalization of a patient to an acute care medical facility by emergency transport, a transition destination at an early stage from initial information on the patient.

### Means to Solve the Problem

A medical information processing system according to one embodiment of this invention comprises,

an input unit configured to receive input of electronic medical chart information on a subject patient;

a machine learning unit configured to machine-learn a transition destination of each patient from an acute care medical facility by referring to an electronic medical chart information group of each patient obtained from inpatients of the acute care medical facility to; and

a transition destination prediction unit configured to predict, based on a learning result obtained by the machine learning unit, the transition destination of the subject patient from the received electronic medical chart information on the subject patient.

A medical information processing method, which is to be performed by a medical information processing system, according to one embodiment of this invention, the medical information processing method comprising:

machine learning, previously, by a machine learning unit, a transition destination of each patient from the acute care medical facility based on an electronic medical chart information group of each patient obtained from inpatients of an acute care medical facility;

receiving, by an input unit, input of electronic medical chart information on a subject patient; and

predicting, by a transition destination prediction unit, based on a learning result obtained by the machine learning unit, the transition destination of the subject patient from the received electronic medical chart information on the subject patient.

A recording medium, according to one embodiment of this invention, causing a processor of an information processing system to operate as:

an input unit configured to receive input of electronic medical chart information on a subject patient;

a machine learning unit configured to machine-learn a transition destination of each patient from an acute care medical facility by referring to an electronic medical chart information group of each patient obtained from inpatients of the acute care medical facility; and

a transition destination prediction unit configured to predict, based on a learning result obtained by the machine learning unit, the transition destination of the subject patient from the received electronic medical chart information on the subject patient.

### Effect of the Invention

According to this invention, the medical information processing system configured to predict, with the hospitalization of the patient to the acute care medical facility by emergency transport, the transition destination at the early stage even from initial information on the patient can be provided.

### BRIEF DESCRIPTION OF THE DRAWING

FIG. 1 is a block diagram for illustrating a medical information processing system 1 according to a first embodiment of this invention.

FIG. 2 is a diagram for explaining an example of a part of items in an electronic medical chart database.

FIG. 3 is a flowchart for illustrating a basic flow of the medical information processing system 1 according to the first embodiment of this invention.

FIG. 4 is a flowchart for illustrating a schematic machine learning flow of the medical information processing system 1 according to the first embodiment of this invention.

FIG. 5 is a flowchart for illustrating a transition destination prediction flow of the medical information processing system 1 according to the first embodiment of this invention.

FIG. 6 is a diagram for explaining operational advantages of the medical information processing system 1 according to the first embodiment of this invention.

FIG. 7 is a block diagram for illustrating a configuration example of a medical information processing system according to at least one embodiment of this invention.

FIG. 8 is a block diagram for illustrating another configuration example of the medical information processing system according to at least one embodiment this invention.

### MODE FOR EMBODYING THE INVENTION

At least one embodiment of this invention is now described with reference to the drawings.

### Embodiment

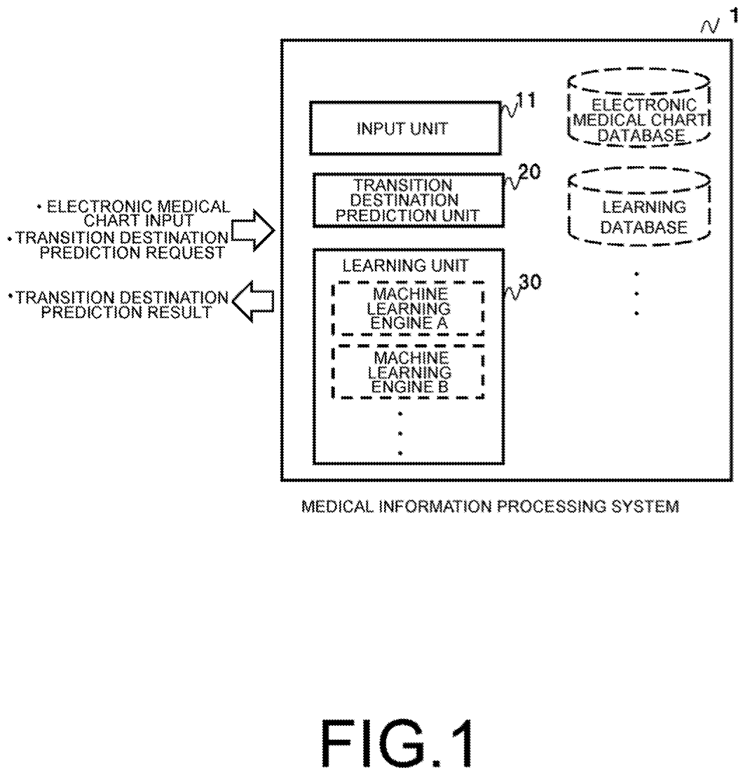

FIG. 1 is a block diagram for illustrating a medical information processing system 1 according to at least one embodiment of this invention.

The medical information processing system 1 includes at least an input unit 11, a transition destination prediction unit 20, and a learning unit 30. In the medical information processing system 1, it is assumed that various databases are constructed in which each constituent element is available as required. The various databases to be used may be an external database instead of an internal database. The medical information processing system 1, which is an information processing system, includes a processor and a memory, and operates each component as follows by a transition destination prediction program in at least one embodiment of this invention.

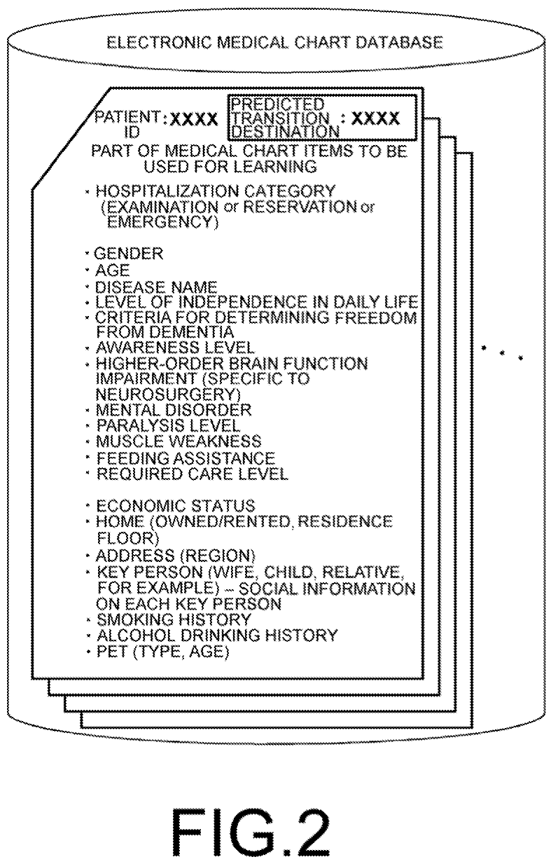

The input unit 11 sequentially receives input of electronic medical chart information on each of patients including a subject patient to be described later, and sequentially registers the received input of electronic medical chart information in an electronic medical chart database. The input unit 11 also receives a transition destination prediction request on the subject patient from a user (such as a doctor or a hospital staff member) or another related program. As illustrated in the figure, the electronic medical chart database may be provided as an internal database in the medical information processing system 1, or an externally provided electronic medical chart system may be used as described above. The electronic medical chart database (system) may also be configured to generate, each time the electronic medical chart information on each patient is updated and stored, a transition destination prediction request for the relevant patient. As items to be managed in the electronic medical chart database, which are not particularly limited, it is possible to use items commonly used in many hospitals. In a case where there is a medical chart item whose data has been accumulated in the past, that item may be added even when the item is unique to the hospital. The structure of the electronic medical chart database is not particularly limited, and an example of the items to be used in information processing in at least one embodiment of this invention is illustrated in FIG. 2.

The transition destination prediction unit 20 predicts, based on the electronic medical chart information on the patient (subject patient) for which a transition destination is to be predicted and a machine learning result accumulated through machine learning performed by the learning unit 30, the transition destination after hospitalization by emergency transport of the subject patient by using the electronic medical chart information on the subject patient as primary information. At this time, the transition destination prediction unit 20 uses subject patient data input for the subject patient having an acute medical condition as the electronic medical chart information on the subject patient to narrow down the transition destination based on a machine learning result obtained from the electronic medical chart information on a patient group used for learning. Electronic medical chart information items include a disease name and medical condition of the subject patient, but not all of the details of the items are required for the prediction. For example, even immediately after the emergency transport or even when the explicit medical condition is unknown, it is desired that the predicted transition destination of the subject patient be determined based on the learning result obtained by performing machine learning on items being blank or items being input as unknown.

The transition destination prediction unit 20 may classify the transition destination of the subject patient as a home or a facility based on which of discharge to the home, transfer to a convalescent hospital (also called a rehabilitation hospital, for example), or transfer to other facility has a higher likelihood. The transition destination prediction unit 20 in at least one embodiment of this invention predicts the transition destination of the subject patient by using one (one type of) machine learning result.

The home classified here is a result predicted that the patient is at a level at which the patient can live at home. Similarly, the convalescent hospital (rehabilitation hospital) classified here is a result predicted that an appropriate rehabilitation environment is to be provided to the patient and the patient may ultimately recover to a level at which the patient can live at home on condition that the patient undergoes rehabilitation. Meanwhile, for the other facility classified here, the patient who may ultimately have difficulty in living at home even after undergoing rehabilitation is classified. The other facilities include, for example, medical institutions such as sanatoriums, health care facilities, and nursing homes.

The above-mentioned classification has three categories, namely, a home, a convalescent hospital (rehabilitation hospital), and other facility, but medical institutions such as a sanatorium may be extracted as a new item from the other facilities, and the classification may have four categories, namely, a home, a convalescent hospital (rehabilitation hospital), other medical facility, and other facility, or more categories.

In a specific example, the transition destination prediction unit 20 acquires the electronic medical chart information on the subject patient acquired at the present time, and outputs, based on the machine learning result classified into multiple classes, any one of a home, a convalescent hospital (rehabilitation hospital), and other facility (sanatorium, rehabilitation facility, or care and nursing facility) as the prediction result.

In another example, when selecting any one of a home, a convalescent hospital (rehabilitation hospital), and other facility as the transition destination of the subject patient, the transition destination prediction unit 20 may select the transition destination from among a rehabilitation facility and a care and nursing facility that satisfy a condition in a case in which a probability that a home is not selectable as the transition destination is higher than a threshold value.

The classification of the transition destination is considered to be strongly influenced by several items registered in the electronic medical chart of the subject patient. For example, items such as the presence or absence of a home, the floor number that the home is on, and whether the home is owned or rented can be parameters that, together with the degree of recovery of the patient, strongly influence whether or not a home is to be the transition destination. Even when a home is the transition destination, the degree of recovery of the patient indicates that the patient is expected to recover in some cases and that recovery is not expected in other cases. Although it is difficult for medical staff to accurately determine those matters at an early stage, a highly accurate prediction result can be provided to the medical staff from an early stage by using the machine learning result of an acute care medical facility.

The learning unit 30 refers to the electronic medical chart database to machine-learn the transition destination of each patient from the acute care medical facility obtained from the inpatients of the acute care medical facility for the electronic medical chart information group of each patient, and accumulates the learning results in a learning database. The learning unit 30 operates as machine learning means. It is desired, but not essential, that the machine learning include the disease name and medical condition in the electronic medical chart information items to be used for the learning. Together with hospitalization category and patient status parameters, it is further desired that, as in the example of items illustrated in FIG. 2, patient characteristic social parameters including economic status, a home, an address, a key person, smoking history, and alcohol drinking history be frequently used for the electronic medical chart information items (parameter group) to be applied in the machine learning. The learning unit 30 machine-learns the transition destination after discharge from hospital for this electronic medical chart information group. The machine learning method is not particularly limited, and there may be used a regression method, for example, a support vector machine (SVM), a clustering method, for example, a k-nearest neighbor method, or a neural network method. For example, as the machine learning method, there may be learned both a machine learning method with a high transition destination accuracy rate when using electronic medical chart data obtained on the hospitalization date of the hospitalization of an emergency transport patient, and a machine learning method with a high transition destination accuracy rate when using electronic medical chart data accumulated up to one week after hospitalization. As described above, it is desired that the learning unit 30 be configured to handle a plurality of learning methods so that the learning data to be used by the transition destination prediction unit 20 can be switched automatically or by a user operation depending on the period after hospitalization.

There are now explained several of the patient characteristic social parameters illustrated in FIG. 2.

The parameter “economic status” is a parameter indicating the economic status of the patient or family sharing the household budget.

The parameter “home” is a parameter indicating whether the house in which the patient or family sharing the household budget lives is owned or rented. This parameter may also include which floor the living space is on.

The parameter “address” is a parameter indicating the region in which the house in which the patient or family sharing the household budget lives is located.

The parameter “key person” is a parameter indicating the presence or absence of a close family member or friend living with the patient, and the relationship with the person. This parameter is simply operated by inputting the cohabiting family structure. The social characteristics of each person registered in this “key person” may be received and added to the machine learning parameters. It can be estimated that, by collecting a large volume of data for this item and using the collected data together with those for other items, it is possible to obtain, as a mining result, a significant difference regarding whether or not home medical care/home rehabilitation/hospital visitation can be performed.

The parameter “smoking history” is a parameter indicating the smoking history of the patient.

The parameter “alcohol drinking history” is a parameter indicating the alcohol drinking history of the patient.

The parameter “pet” indicates the type and age of a pet raised by the patient or family living with the patient. The rearing years or the like may be included.

The items in the electronic medical chart database illustrated in FIG. 2 are merely examples, and the items in the electronic medical chart database are not limited to those illustrated in the figure. In the electronic medical chart database, various items may be freely added or changed, and the resultant items may be used as parameters. In particular, the addition of patient characteristic social parameters and the addition of regionality to the electronic medical chart database can be beneficial for the machine learning of the transition destination.

Naturally, as the treatment of the patient progresses, the level of certainty of the relationship between the transition destination and each parameter of each patient becomes higher.

By the above-mentioned configuration, the medical information processing system 1 can predict, with the hospitalization of a patient to an acute care medical facility by emergency transport, the transition destination of the patient at an early stage.

### Explanation of Operation in Embodiment

Next, an operation of the medical information processing system 1 according to at least one embodiment of this invention is explained.

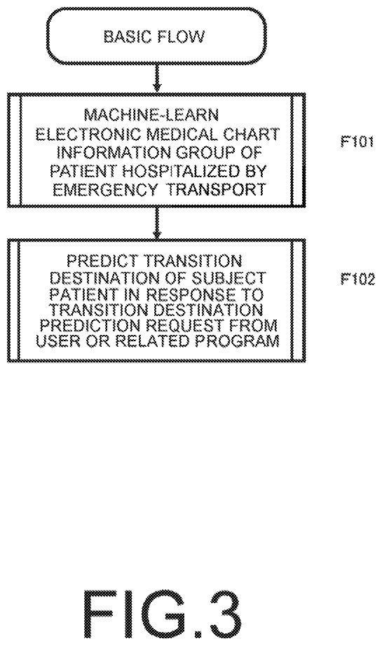

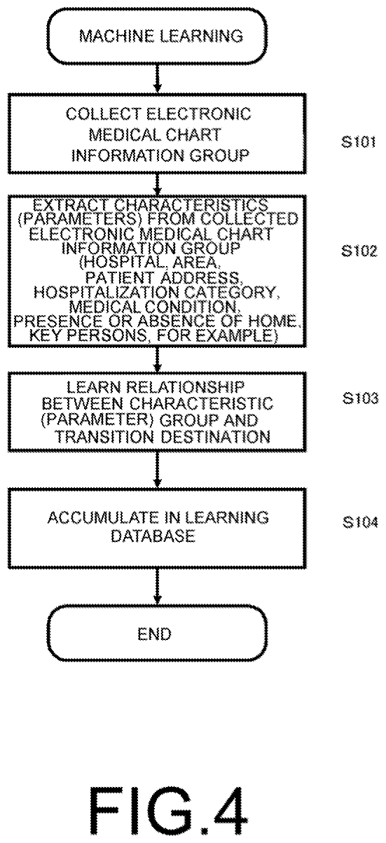

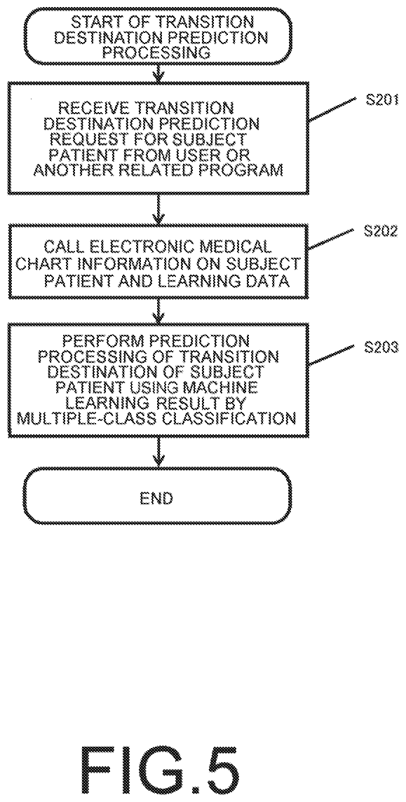

FIG. 3 is a flowchart for illustrating a basic flow of the medical information processing system 1 according to at least one embodiment. FIG. 4 is a flowchart for illustrating the machine learning flow of the medical information processing system 1. FIG. 5 is an example of a flowchart for illustrating the transition destination prediction flow of the medical information processing system 1.

Firstly, the basic flow is as follows.

The medical information processing system 1 causes the learning unit 10 to previously machine-learn the transition destination after hospitalization by emergency transport regarding the electronic medical chart information group of each patient (Step F101).

The medical information processing system 1 causes the transition destination prediction unit 20 to sequentially predict the transition destination of the subject patient based on both the result of machine learning and the electronic medical chart information on the subject patient input with the hospitalization of the subject patient by emergency transport (Step F102).

As in this flow, the medical information processing system 1 appropriately receive a transition destination prediction request from a person or another program, and can predict the transition destination by using the electronic medical chart information on the subject patient at the receiving time as primary information. From this prediction, based on the transition destination that is the prediction result, a user (e.g., doctor, nurse, or social worker) being a request source can sequentially know the transition destination predicted based on the current input information. The knowing of the transition destination is made possible immediately after the creation of the electronic medical chart information on the subject patient immediately after emergency transport.

FIG. 4 is an example of a flowchart for illustrating the machine learning flow of the medical information processing system 1.

First, the processor of the information processing system serving as the medical information processing system 1 sequentially collects the electronic medical chart information group to be learned (Step S101).

Next, the processor extracts the data of the electronic medical chart items (characteristics and parameters) to be learned from the collected electronic medical chart information group (Step S102). The characteristics include the hospital, the region, the patient address, the hospitalization category, presence or absence of a home, and key persons, as well as the medical condition and the disease name of the patient.

Next, the processor learns the relationship between the characteristic (parameter) group and the transition destination (Step S103).

Lastly, the processor accumulates the learning result in the learning database (Step S104).

It is desired that the machine learning be performed regularly and updated to the latest learning results.

FIG. 5 is an example of a flowchart for illustrating the transition destination prediction flow of the medical information processing system 1.

The processor of the information processing system serving as the medical information processing system 1 receives a transition destination prediction request for a subject patient from a user or another related program (Step S201).

Next, the processor calls the current electronic medical chart information on the subject patient and the learning data (Step S202).

Next, the processor performs prediction processing of the transition destination of the subject patient based on the machine learning result (Step S203). This prediction processing may be performed by, for example, classifying, based on the learning result, the current electronic medical chart information on the subject patient into multiple classes including discharge from hospital, transfer to a convalescent hospital (rehabilitation hospital), and other facilities as classification candidates.

Lastly, the processor notifies the request source of the transition destination and the like (Step S204).

The transition destination prediction processing is appropriately performed in response to a request from a user or the like via the input unit 11. By operating the information processing system in this way, with the hospitalization of a patient by emergency transport, the medical information processing system 1 can predict the transition destination of the patient at an early stage. Further, this prediction is sequentially and quickly improved with high accuracy as a result of the input to the electronic medical chart of the patient medical condition and various information sequentially input from the initial information.

Operational advantages of the medical information processing system 1 are now explained.

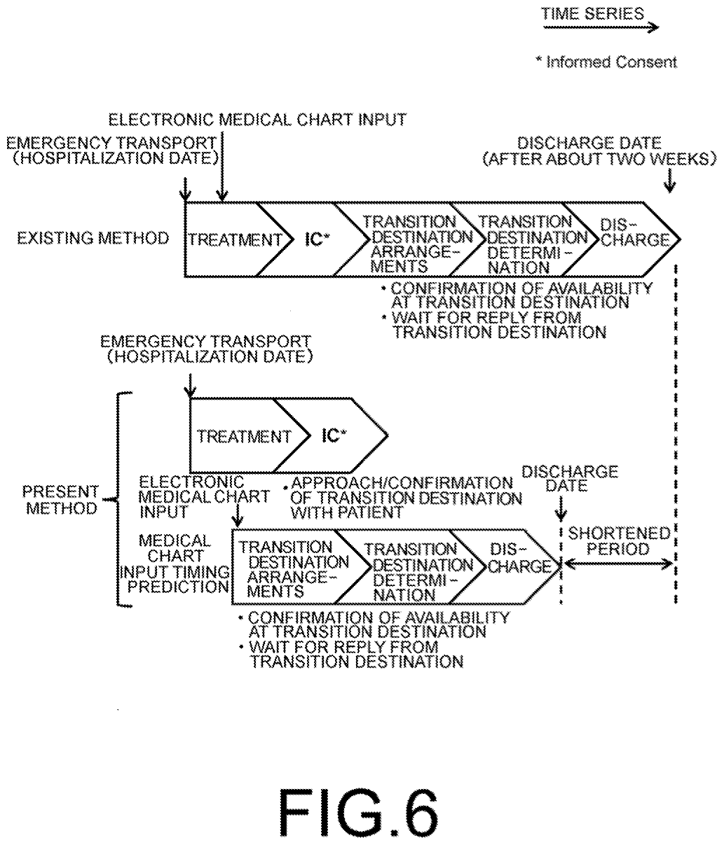

FIG. 6 is a diagram for visually explaining the operational advantages of the medical information processing system 1.

As in the existing method illustrated in the figure, a patient who has been hospitalized by emergency transport in an existing medical facility is provided with an established flow until transfer in which the patient undergoes a routine in order of “treatment”→“informed consent”→“transition destination arrangements”→“transfer destination determination”→“discharge from hospital (move to the transition destination)”.

Referring to statistical data, many inpatients in the emergency department can be transitioned from the acute care medical facility in about 14 days (two weeks). At present, for patients having a medical condition that allows them to be transitioned, transition is performed after a negotiation between the person in charge at the receiving facility and the person in charge at the acute care medical facility. Meanwhile, for some patients, their transition from the acute care medical facility is delayed as a result of factors such as availability at the receiving facility.

However, through use of the method of this invention, for example, a social worker or the like can know the predicted transition destination categorized from the early-stage electronic medical chart information on the subject patient (even from the initial information, for example). As a result, staff such as social workers can start transfer destination arrangements to other hospitals and facilities and various preparations at an early stage. This is because, for example, in contrast to performing transfer destination arrangements after informed consent as in the existing method, the work flow in the hospital of transfer destination arrangements and the like can be performed in parallel. As a result, transfer can be performed at an early stage once the patient has recovered. For patients, there is a benefit in that the patient can shift at an early stage from treatment at an acute care hospital to treatment, for example, rehabilitation. For patients and the medical system, medical expenses can be reduced by optimizing the length of hospital stay. For acute care hospitals, there is also a benefit in that cases in which there are not enough hospital beds due to patients who have recovered staying longer than required can be reduced.

For patients, whether to be transferred to a convalescent hospital (rehabilitation hospital) or to move to other facility is a big decision to make because it may in the end lead to permanently entering a facility without returning home. However, accurate early prediction of this important decision is also very difficult for medical institution workers.

Through sequential addition to the electronic medical chart information, transition destination arrangements can be performed based on an early prediction. Further, it is also possible to execute an early patient discharge plan by performing transition destination arrangements ahead of schedule.

As explained above, the medical information processing system to which this invention is applied can predict, with the hospitalization of a patient to an acute care medical facility by emergency transport, the transition destination at an early stage even from the initial information on the patient.

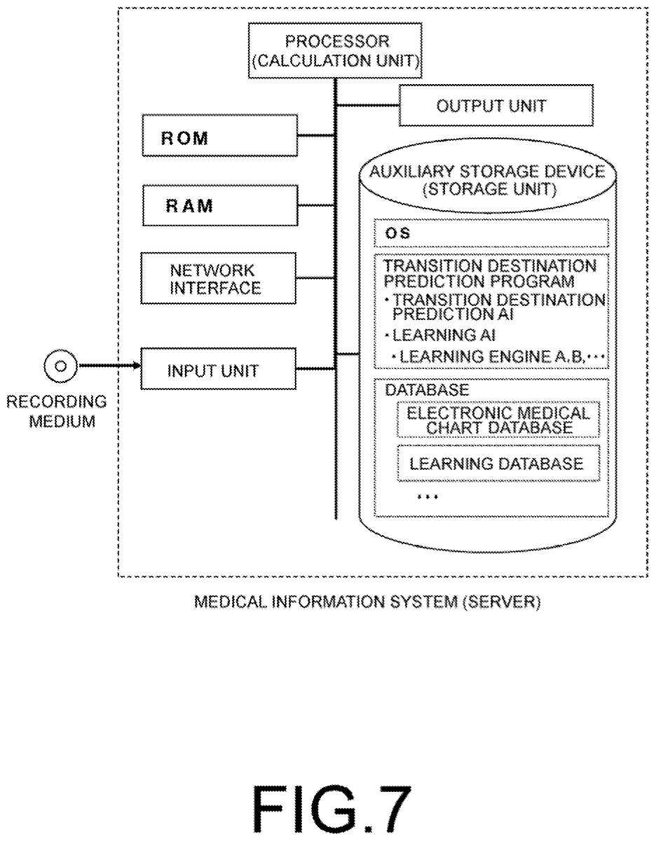

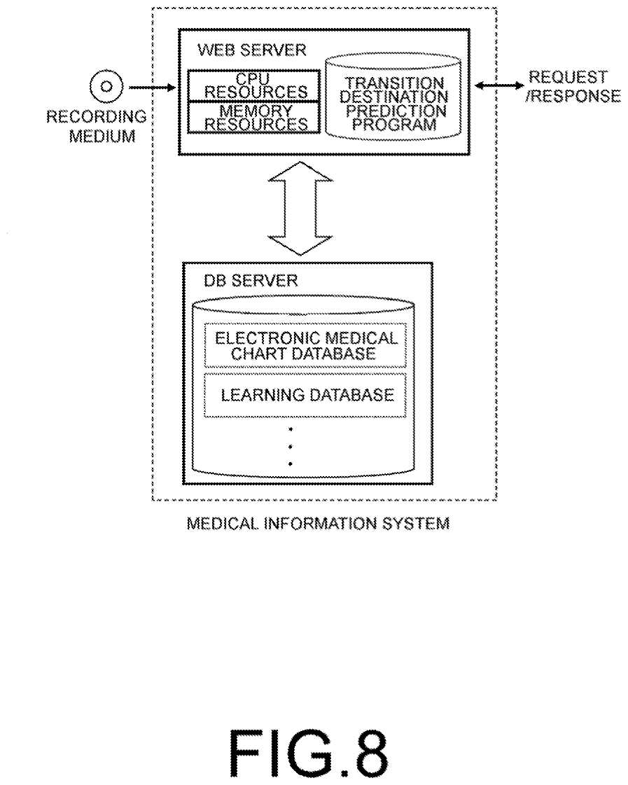

Each part of the system may be implemented by appropriately using a combination of the hardware and software of a computer system (server system) and virtualization technology, as illustrated in FIG. 7 and FIG. 8. The computer system includes one or more processors and memories tailored to the desired mode. In this computer system mode, each part may be implemented by a guidance system program developed in the memory, in which hardware, for example, one or more processors is operated by an execution instruction group or a code group based on the program. In this case, the program may implement each part in cooperation with functions provided by software, such as an operating system, a microprogram, and a driver, as required.

The program data developed in the memory includes as appropriate an execution instruction group, a code group, a table file, content data, and the like that cause the processor to operate as one or more of the above-mentioned units.

The computer system is not required to be constructed as a single device, and may be constructed as a so-called thin client, distributed computing, or cloud computing by combining a plurality of servers, computers, virtual machines, and the like.

A part or all of the computer system may be replaced with hardware or firmware (e.g., one or more of large-scale integration: LSI, field programmable gate array: FGPA, a combination of electronic elements). Similarly, only a portion of each part may be replaced with hardware or firmware.

The program may be recorded in a non-transitory manner on a recording medium and distributed. The program recorded on the recording medium is read into the memory in a wired manner, in a wireless manner, or via the recording medium itself, and operates the processor and the like.

In this specification, the term “recording medium” includes similarly-termed storage media, memory devices, storage devices, and the like. Examples of the recording medium include an optical disc, a magnetic disk, a semiconductor memory device, a hard disk device, and a tape medium. It is desired that the recording medium be non-volatile. The recording medium may be a combination of a volatile module (e.g., random access memory (RAM)) and a nonvolatile module (e.g., read only memory (ROM)).

Stating the above-mentioned embodiment another way, the medical information processing system according to at least one embodiment of this invention can be implemented by causing an information processing system configured to operate as the medical information processing system to operate as an input unit, a learning unit, and a transition destination prediction unit based on a transition destination prediction program developed in a memory.

Similarly, stating the above-mentioned embodiment another way, the medical information processing system according to at least one embodiment of this invention can be constructed by a recording medium including a transition destination prediction program configured to be expanded in a memory and be operated by a processor of the information processing system, the recording medium being configured to cause an information processing resource to execute a learning step, an input step, and a transition destination prediction step in a timely manner.

At least one embodiment of this invention is described as an example. However, specific configurations of this invention are not limited to the above-mentioned embodiment, and changes without departing from the gist of the invention are also included in this invention. For example, changes such as separation and merging of the block components and a switch of processing steps in the above-mentioned embodiment can be freely carried out as long as the purport and the above-mentioned functions of this invention are satisfied, and the above description does not limit this invention.

This application is based upon and claims the benefit of priority from Japanese patent application No. 2017-165607, filed on Aug. 30, 2017, the disclosure of which is incorporated herein in its entirety by reference.

### REFERENCE SIGNS LIST

1 medical information processing system (computer system)

11 input unit

20 transition destination prediction unit

30 learning unit

1. A medical information processing system, comprising:
an input unit configured to receive input of electronic medical chart information on a subject patient;
a machine learning unit configured to machine-learn a transition destination of each patient from an acute care medical facility by referring to an electronic medical chart information group of each patient obtained from inpatients of the acute care medical facility; and
a transition destination prediction unit configured to predict, using a learning result obtained by the machine learning unit, the transition destination of the subject patient from the received electronic medical chart information on the subject patient.

2. The medical information processing system according to claim 1, wherein the transition destination prediction unit is configured to receive the electronic medical chart information on the subject patient, and to select, by using the learning result, the transition destination of the subject patient based on which of discharge to a home, transfer to a convalescent hospital, and transfer to other facility has a higher likelihood.

3. The medical information processing system according to claim 1, wherein the transition destination prediction unit is configured to select, when selecting any one of a home, a convalescent hospital, and other facility as the transition destination of the subject patient, a convalescent hospital or other facility that satisfies a condition in a case in which a probability that a home is not selectable as the transition destination is higher than a threshold value.

4. The medical information processing system according to claim 1, wherein the transition destination prediction unit is configured to receive, at least data on a family structure living with a patient in the electronic medical chart information for a patient having emergency as a hospitalization category input as the electronic medical chart information on the subject patient, to predict, using the learning result, the transition destination after hospitalization by emergency transport of the subject patient, and to classify the transition destination as discharge to a home, transfer to a convalescent hospital, or transfer to other facility.

5. A medical information processing method, comprising:
machine learning, a transition destination of each patient from the acute care medical facility based on an electronic medical chart information group of each patient obtained from inpatients of an acute care medical facility;
receiving input of electronic medical chart information on a subject patient; and
predicting, using a machine learning result, the transition destination of the subject patient from the received electronic medical chart information on the subject patient.

6. The medical information processing method according to claim 5, wherein the predicting of the transition destination includes receiving the electronic medical chart information on the subject patient, and selecting, using the machine learning result the transition destination of the subject patient based on which of discharge to a home, transfer to a convalescent hospital, and transfer to another facility has a higher likelihood.

7. The medical information processing method according to claim 5, wherein, the predicting of the transition destination includes selecting when selecting any one of a home, a convalescent hospital, and other facility as the transition destination of the subject patient, a convalescent hospital or other facility that satisfies a condition in a case in which a probability that a home is not selectable as the transition destination is higher than a threshold value.

8. The medical information processing method according to claim 5, wherein the predicting of the transition destination includes receiving at least data on a cohabiting family structure in the electronic medical chart information for a patient having emergency as a hospitalization category input as the electronic medical chart information on the subject patient, predicting, using the learning result, the transition destination after hospitalization by emergency transport of the subject patient, and classifying the transition destination as discharge to a home, transfer to a convalescent hospital, or transfer to other facility.

9. A recording medium having stored thereon, in a non-transitory manner, a program for causing a processor of an information processing system to operate as:
an input unit configured to receive input of electronic medical chart information on a subject patient;
a machine learning unit configured to machine-learn a transition destination of each patient from an acute care medical facility by referring to an electronic medical chart information group of each patient obtained from inpatients of the acute care medical facility; and
a transition destination prediction unit configured to predict, using a learning result obtained by the machine learning unit, the transition destination of the subject patient from the received electronic medical chart information on the subject patient.

10. The recording medium according to claim 9, wherein the processor is caused to operate so as to receive the electronic medical chart information on the subject patient, and to select, using the learning result, the transition destination of the subject patient based on which of discharge to a home, transfer to a convalescent hospital, and transfer to other facility has a higher likelihood.

11. The recording medium according to claim 9, wherein the processor is caused to operate so as to select, when selecting any one of a home, a convalescent hospital, and other facility as the transition destination of the subject patient, a convalescent hospital or other facility that satisfies a condition in a case in which a probability that a home is not selectable as the transition destination is higher than a threshold value.

12. The recording medium according to claim 9, wherein the processor is caused to operate so as to receive, at least data on a family structure living with a patient in the electronic medical chart information for a patient having emergency as a hospitalization category input as the electronic medical chart information on the subject patient, to predict, using the learning result, the transition destination after hospitalization by emergency transport of the subject patient, and to classify the transition destination as discharge to a home, transfer to a convalescent hospital, or transfer to other facility.
